(19) United States
(12) Patent Application Publication
(10) Pub. No.: US 2025/0072827 A1
(43) Pub. Date: Mar. 6, 2025

(54) THERMAL SYSTEM WITH PATIENT SENSOR(S)

(71) Applicant: Stryker Corporation, Portage, MI (US)

(72) Inventors: Gregory S. Taylor, Kalamazoo, MI (US); Marko N. Kostic, Oshawa (CA); Marco Constant, Portage, MI (US); Christopher John Hopper, Kalamazoo, MI (US); Anuj K. Sidhu, Kalamazoo, MI (US)

(21) Appl. No.: 18/952,201

(22) Filed: Nov. 19, 2024

Related U.S. Application Data
(62) Division of application No. 18/226,062, filed on Jul. 25, 2023.
Application No. 18/226,062 is a continuation of application No. 16/957,809, filed on Jun. 25, 2020, now abandoned.
(60) Provisional application No. 62/610,327, filed on Dec. 26, 2017.

(51) Int. Cl. A61B 5/00; A61B 5/01; A61B 5/026; A61B 5/053; A61B 8/08; A61F 7/00; A61F 7/02

(52) U.S. Cl. CPC A61B 5/4836; A61F 7/0085; A61F 7/02; A61B 5/0075; A61B 5/01; A61B 5/026; A61B 5/053; A61B 5/6892; A61B 8/08; A61F 2007/0054; A61F 2007/0086; A61F 2007/0093; A61F 2007/0096

(57) ABSTRACT

A thermal control unit supplies temperature controlled fluid to a patient to control the patient's temperature. The thermal control unit includes a fluid outlet, fluid inlet, heat exchanger, pump, patient core temperature probe port, auxiliary sensor port, and controller. The controller receives patient core temperature readings from the patient temperature probe port and auxiliary sensor readings from the auxiliary sensor port. The controller may control a temperature of the circulating fluid in both a feedback manner using patient core temperature readings and a feedforward manner using readings from the auxiliary sensor. The auxiliary sensor may measure a characteristic of the patient's tissue indicative of thermal resistance, and/or the auxiliary sensor may measure a temperature of the patient's tissue at an intermediate depth. The controller may use the intermediate temperature to predict arrival at a target patient temperature. The auxiliary sensor may be an ultrasonic sensor, infrared sensor, or the like.

### CROSS-REFERENCE TO RELATED APPLICATIONS

This application is a divisional of, and claims priority to, U.S. patent application Ser. No. 18/226,062 filed Jul. 25, 2023, by inventors Gregory S. Taylor et al. and entitled THERMAL SYSTEM WITH PATIENT SENSOR(S), which in turn claims priority to U.S. patent application Ser. No. 16/957,809 filed Jun. 25, 2020, by inventors Gregory S. Taylor et al. and entitled THERMAL SYSTEM WITH PATIENT SENSORS(S), which is § 371 application of PCT/US2018/066114 filed Dec. 18, 2018, which in turn claims priority to U.S. provisional patent application Ser. No. 62/610,327 filed Dec. 26, 2017, by inventors Gregory S. Taylor et al. and entitled THERMAL SYSTEM WITH PATIENT SENSORS, the complete disclosures of all of which are incorporated herein by reference.

### BACKGROUND

The present disclosure relates to a thermal control system for controlling the temperature of circulating fluid that is delivered to one or more thermal pads positioned in contact with a patient.

Thermal control systems are known in the art for controlling the temperature of a patient by providing a thermal control unit that supplies temperature controlled fluid to one or more thermal pads positioned in contact with a patient. The thermal control unit includes one or more heat exchangers for controlling the temperature of the fluid and a pump that pumps the temperature controlled fluid to the pad(s). After passing through the pad(s), the fluid is returned to the thermal control unit where any necessary adjustments to the temperature of the returning fluid are made before being pumped back to the pad(s).

In some instances, the temperature of the fluid is controlled to a static target temperature, while in other instances the temperature of the fluid is varied as necessary in order to automatically effectuate a target patient temperature. When the thermal control unit automatically controls the temperature of the circulating fluid in order to effectuate a desired patient temperature, the thermal control unit utilizes patient temperature measurements in a closed-loop feedback manner. The closed loop feedback gives the thermal control unit knowledge of the patient's temperature, which it uses to determine whether to heat or cool the circulating fluid, or to maintain the circulating fluid at its current temperature. In some instances, the closed-loop feedback control of the temperature of the circulating fluid causes the patient's temperature to overshoot its target temperature, both when the patient's temperature initially reaches the target temperature and during subsequent arrivals at the target temperature.

### SUMMARY

According to one or more of the various embodiments disclosed herein, one or more sensors are utilized during the thermal therapy of a patient in order to better determine a particular patient's responsiveness to the thermal treatment, and to use that thermal responsiveness so as to reduce overshoot in the patient's temperature and/or more quickly bring the patient's temperature to a target temperature. In some embodiments, the patient sensor(s) are used to measure a characteristic of the patient's morphology and to use that morphology to predict how resistive the patient will be to having his or her core temperature adjusted to a target temperature. In some embodiments, the patient sensor(s) are used to measure a temperature of the patient at a depth intermediate the patient's skin and the patient's core. Monitoring changes in the intermediate temperature relative to an externally applied temperature (e.g. via thermal pads) allows a controller to predict when the patient's core temperature will arrive at a target temperature and/or to more accurately control the patient's temperature, including reductions in temperature overshoot. These and other aspects of the various embodiments are discussed in more detail below.

According to one embodiment of the present disclosure, a thermal control unit is provided for controlling a patient's temperature. The thermal control unit includes a fluid outlet, a fluid inlet, a circulation channel, a pump, a heat exchanger, a fluid temperature sensor, a patient temperature probe port, an auxiliary sensor port, and a controller. The fluid outlet is adapted to fluidly couple to a fluid supply line of a thermal pad, and the thermal pad is adapted to be wrapped around a portion of the patient's body. The fluid inlet is fluidly coupled to a fluid return line of the thermal pad. The circulation channel couples the fluid inlet to the fluid outlet. The pump circulates fluid through the circulation channel from the fluid inlet to the fluid outlet. The heat exchanger is adapted to add or remove heat from the fluid circulating in the circulation channel. The fluid temperature sensor senses a temperature of the fluid and the patient temperature probe port receives patient core temperature readings from a patient temperature probe. The auxiliary sensor port receives sensor readings from a non-temperature sensor adapted to detect a patient parameter. The controller communicates with the patient temperature probe port, the pump, the fluid temperature sensor, and the auxiliary sensor port. The controller controls the heat exchanger based on both the patient core temperature readings and the sensor readings from the non-temperature sensor.

According to other aspects of the present disclosure, the non-temperature sensor includes one or more of the following: a bio-impedance sensor adapted to detect electrical impedance of the patient; an ultrasonic sensor adapted to detect attenuation levels of ultrasonic waves traveling through at least a portion of the patient's body; a near infrared sensor adapted to detect attenuation levels of near infrared waves traveling through at least a portion of the patient's body; a perfusion sensor adapted to detect a patient's blood perfusion levels, and an end tidal carbon dioxide (ETCO₂) sensor adapted to measure carbon dioxide levels in air exhaled from the patient. If implemented as a perfusion sensor, the perfusion sensor is positioned to detect the patient's blood perfusion levels at the patient's palm and/or foot, in at least some embodiments.

The controller is adapted, in at least one embodiment, to use the sensor readings from the non-temperature sensor to estimate a thickness of the patient's body. The estimate may involve assigning a Body Mass Index (BMI) category to the patient that encompasses multiple Body Mass Indexes, rather than an individual BMI reading.

In some embodiments, the one or more non-temperature sensors are integrated into thermal pad.

The controller may use a first set of coefficients to control the heat exchanger when the sensor readings from the non-temperature sensor meet a first criteria and use a second set of coefficient to control the heat exchanger when the sensor readings from the non-temperature sensor meet a second criteria. The criteria includes a thickness of the patient's body at one or more locations, in some embodiments.

According to some aspects, the controller estimates a level of patient resistance to thermal treatment based on the sensor readings from the non-temperature sensor and uses the estimated level of resistance to thermal treatment when controlling the heat exchanger.

The controller may use the patient parameter to control the heat exchanger in a feedforward manner.

According to another embodiment of the present disclosure, a thermal control unit is provided for controlling a patient's temperature. The thermal control unit includes a fluid outlet, a fluid inlet, a circulation channel, a pump, a heat exchanger, a fluid temperature sensor, a patient temperature probe port, a patient temperature sensor port, and a controller. The fluid outlet is adapted to fluidly couple to a fluid supply line of a thermal pad, and the thermal pad is adapted to be wrapped around a portion of the patient's body. The fluid inlet is fluidly coupled to a fluid return line of the thermal pad. The circulation channel couples the fluid inlet to the fluid outlet. The pump circulates fluid through the circulation channel from the fluid inlet to the fluid outlet. The heat exchanger is adapted to add or remove heat from the fluid circulating in the circulation channel. The fluid temperature sensor senses a temperature of the fluid and the patient temperature probe port receives patient core temperature readings from a patient temperature probe. The patient temperature sensor port receives intermediate temperature readings from a patient temperature sensor positioned at an exterior location of the patient's body. The patient temperature sensor measures a temperature of the patient at an intermediate depth between the patient's skin and the patient's core. The controller controls the heat exchanger based on both the patient core temperature readings and the intermediate temperature readings from the patient temperature sensor.

According to other aspects of the present disclosure, the patient temperature sensor is an acoustic thermometer adapted to measure a subcutaneous temperature of the patient.

In some embodiments, the controller monitors a lag time between external application of a cold temperature to the patient's skin via the thermal pad and propagation of the cold temperature to the intermediate depth. The controller may use the lag time to determine when to transition from cooling the fluid to heating the fluid, and/or to reduce overshoot of the patient's temperature beyond a patient target temperature.

In some embodiments, the controller is informed of a value of the intermediate depth when controlling the heat exchanger.

The controller may further be adapted to control the heat exchanger such that the patient core temperature readings move toward a target patient temperature, and the controller uses the lag time to predict when the patient core temperature readings will reach the target patient temperature.

According to another embodiment of the present disclosure, a thermal control unit is provided for controlling a patient's temperature. The thermal control unit includes a fluid outlet, a fluid inlet, a circulation channel, a pump, a heat exchanger, a fluid temperature sensor, a patient temperature probe port, an auxiliary sensor port, and a controller. The fluid outlet is adapted to fluidly couple to a fluid supply line of a thermal pad, and the thermal pad is adapted to be wrapped around a portion of the patient's body. The fluid inlet is fluidly coupled to a fluid return line of the thermal pad. The circulation channel couples the fluid inlet to the fluid outlet. The pump circulates fluid through the circulation channel from the fluid inlet to the fluid outlet. The heat exchanger is adapted to add or remove heat from the fluid circulating in the circulation channel. The fluid temperature sensor senses a temperature of the fluid and the patient temperature probe port receives patient core temperature readings from a patient temperature probe. The auxiliary sensor port receives sensor readings from an auxiliary sensor. The controller controls the heat exchanger based on both the patient core temperature readings and the sensor readings, and the controller uses the sensor readings to control the heat exchanger in a feedforward manner.

According to other aspects, the controller uses the patient core temperature readings as feedback for controlling the heat exchanger.

The auxiliary sensor may include one or more of the following: (1) a bio-impedance sensor adapted to detect electrical impedance of the patient; (2) an ultrasonic sensor adapted to detect attenuation levels of ultrasonic waves traveling through at least a portion of the patient's body; (3) a near infrared sensor adapted to detect attenuation levels of near infrared waves traveling through at least a portion of the patient's body; (4) a perfusion sensor adapted to detect a patient's blood perfusion levels, and (5) an end tidal carbon dioxide (ETCO₂) sensor adapted to measure carbon dioxide levels in air exhaled from the patient.

In some embodiments, the controller uses the auxiliary sensor to estimate a Body Mass Index level of the patient.

The controller may be adapted to estimate a level of patient resistance to thermal treatment based on the sensor readings from the auxiliary sensor. When so adapted, the controller uses the estimated level of resistance to thermal treatment when controlling the heat exchanger.

The auxiliary sensor, in some embodiments, is an intermediate temperature sensor positioned at an exterior location of the patient's body and adapted to measure a temperature of the patient at an intermediate depth between the patient's skin and the patient's core. The controller uses the intermediate temperature sensor, in some embodiments, to monitor a lag time between external application of a cold temperature to the patient's skin via the thermal pad and propagation of the cold temperature to the intermediate depth.

The lag time is used in one or more different manners, such as, but not limited to: determining when to transition from cooling the fluid to heating the fluid (and/or vice versa), and/or predicting when the patient core temperature readings will reach a target patient temperature.

Before the various embodiments disclosed herein are explained in detail, it is to be understood that the claims are not to be limited to the details of operation or to the details of construction, nor to the arrangement of the components set forth in the following description or illustrated in the drawings. The embodiments described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a thermal control system according to one aspect of the present disclosure shown applied to a patient on a patient support apparatus;

FIG. 2 is a block diagram of the thermal control system and thermal control unit of FIG. 1;

FIG. 3 is an illustrative control loop diagram followed in at least one embodiment of the thermal control unit of FIG. 2;

FIG. 4 is a graph of several illustrative responses of patients having different body morphologies to thermal treatment; and

FIG. 5 is a graph of an illustrative set of patient core, peripheral, and intermediate temperature readings during thermal treatment.

### DETAILED DESCRIPTION OF THE EMBODIMENTS

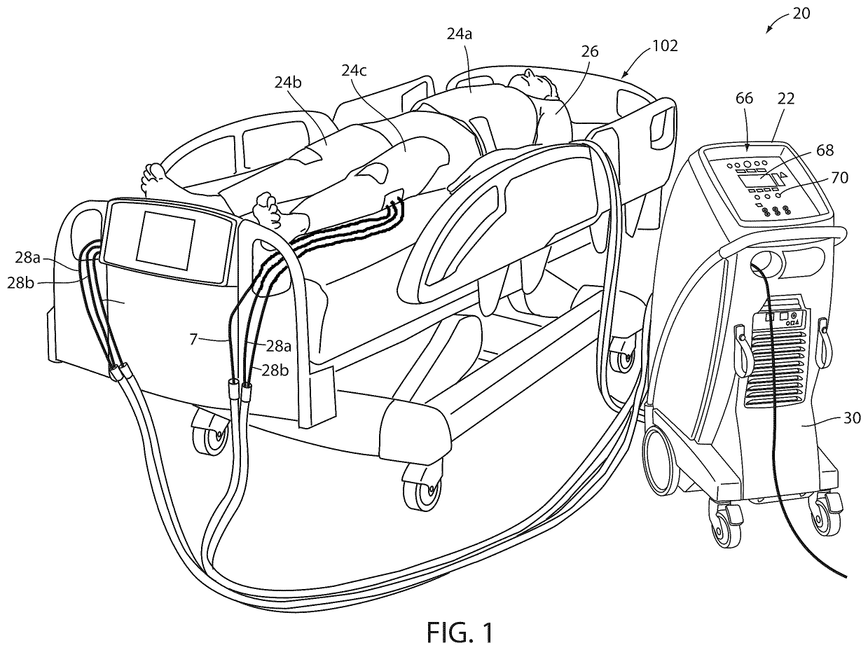

A thermal control system 20 according to one embodiment of the present disclosure is shown in FIG. 1. Thermal control system 20 includes a thermal control unit 22 coupled to one or more thermal therapy devices 24. Thermal control system 20 is adapted to control the temperature of a patient 26, which may involve raising, lowering, and/or maintaining the patient's temperature. The thermal therapy devices 24 are illustrated in FIG. 1 to be thermal pads, but it will be understood that thermal therapy devices 24 may take on other forms, such as, but not limited to, blankets, vests, patches, caps, or other structures that receive temperature-controlled fluid. For purposes of the following written description, thermal therapy devices 24 will be referred to as thermal pads 24, but it will be understood by those skilled in the art that this terminology is used merely for convenience and that the phrase “thermal pad” is intended to cover all of the different variations of thermal therapy devices 24 mentioned above (e.g. blankets, vests, patches, caps, etc.) and variations thereof.

Thermal control unit 22 is coupled to thermal pads 24 via a plurality of hoses 28. Thermal control unit 22 delivers temperature-controlled fluid (such as, but not limited to, water or a water mixture) to the thermal pads 24 via the fluid supply hoses 28a. After the temperature-controlled fluid has passed through thermal pads 24, thermal control unit 22 receives the temperature-controlled fluid back from thermal pads 24 via the return hoses 28b.

In the embodiment of thermal control system 20 shown in FIG. 1, three thermal pads 24 are used in the treatment of patient 26. A first thermal pad 24a is wrapped around a patient's torso, while second and third thermal pads 24b, 24c are wrapped, respectively, around the patient's right and left legs. Other configurations can be used and different numbers of thermal pads 24 may be used with thermal control unit 22, depending upon the number of inlet and outlet ports that are included with thermal control unit 22. By controlling the temperature of the fluid delivered to thermal pads 24 via supply hoses 28a, the temperature of the patient 26 can be controlled via the close contact of the pads 24 with the patient 26 and the resultant heat transfer therebetween.

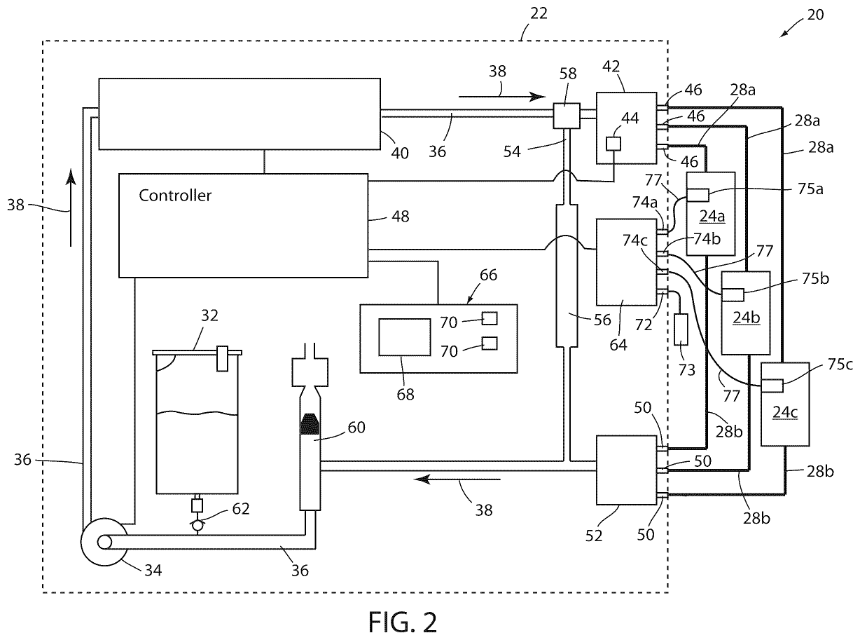

Thermal control unit 22 includes a main body 30 (FIG. 1) to which a removable reservoir 32 may be coupled and uncoupled (FIG. 2). Removable reservoir 32 is configured to hold the fluid that is to be circulated through thermal control unit 22 and the one or more thermal pads 24. By being removable from thermal control unit 22, reservoir 32 can be easily carried to a sink or faucet for filling and/or dumping of the water or other fluid. This allows users of thermal control system 20 to more easily fill thermal control unit 22 prior to its use, as well as to drain thermal control unit 22 after use.

Thermal control unit 22 also includes a pump 34 for circulating fluid through a circulation channel 36 (FIG. 2). Pump 34, when activated, circulates the fluid through circulation channel 36 in the direction of arrows 38 (clockwise in FIG. 2). Starting at pump 34 the circulating fluid first passes through a heat exchanger 40 that adjusts, as necessary, the temperature of the circulating fluid. Heat exchanger 40 may take on a variety of different forms. In some embodiments, heat exchanger 40 is a thermoelectric heater and cooler. In other embodiments, heat exchanger 40 includes a separate chiller and heater. The chiller may take on a variety of different forms, such as, but not limited to, a conventional vapor-compression refrigeration unit having a compressor, condenser, evaporator, expansion valve, and other known components. The heater may also take on a variety of different forms, such as, but not limited to, an electrical resistance heater. Other types of chillers and/or heaters may be used.

After passing through heat exchanger 40, the circulating fluid is delivered to an outlet manifold 42 having an outlet temperature sensor 44 and a plurality of outlet ports 46. Temperature sensor 44 is adapted to detect a temperature of the fluid inside of outlet manifold 42 and report it to a controller 48. Outlet ports 46 are coupled to supply hoses 28a. Supply hoses 28a are coupled, in turn, to thermal pads 24 and deliver temperature-controlled fluid to the thermal pads 24. The temperature-controlled fluid, after passing through the thermal pads 24, is returned to thermal control unit 22 via return hoses 28b. Return hoses 28b couple to a plurality of inlet ports 50. Inlet ports 50 are fluidly coupled to an inlet manifold 52 inside of thermal control unit 22.

It will be understood that, in the embodiment illustrated in FIG. 2, thermal control unit 22 delivers temperature-controlled fluid to outlet manifold 42 that is at a single temperature. In this embodiment, the fluid delivered to each thermal pad 24 has the same temperature. Thermal control unit 22, however, can be modified so that the temperature of the fluid delivered to one or more of the thermal pads 24 can be controlled independently. One example of a such a modification is disclosed in FIG. 9 of commonly assigned U.S. patent application Ser. No. 62/610,362 filed Dec. 26, 2017, by inventor Gregory S. Taylor and entitled THERMAL SYSTEM WITH GRAPHICAL USER INTERFACE, the complete disclosure of which is incorporated herein by reference. Other modifications may also be made to allow independent temperature control of the fluid supplied to thermal pads 24a-c.

Thermal control unit 22 also includes a bypass line 54 fluidly coupled to outlet manifold 42 and inlet manifold 52 (FIG. 2). Bypass line 54 allows fluid to circulate through circulation channel 36 even in the absence of any thermal pads 24 or hoses 28a being coupled to any of outlet ports 46. In the illustrated embodiment, bypass line 54 includes an optional filter 56 that is adapted to filter the circulating fluid. If included, filter 56 may be a particle filter adapted to filter out particles within the circulating fluid that exceed a size threshold, or filter 56 may be a biological filter adapted to purify or sanitize the circulating fluid, or it may be a combination of both. In some embodiments, filter 56 is constructed and/or positioned within thermal control unit 22 in any of the manners disclosed in commonly assigned U.S. patent application Ser. No. 62/404,676 filed Oct. 11, 2016, by inventors Marko Kostic et al. and entitled THERMAL CONTROL SYSTEM, the complete disclosure of which is incorporated herein by reference.

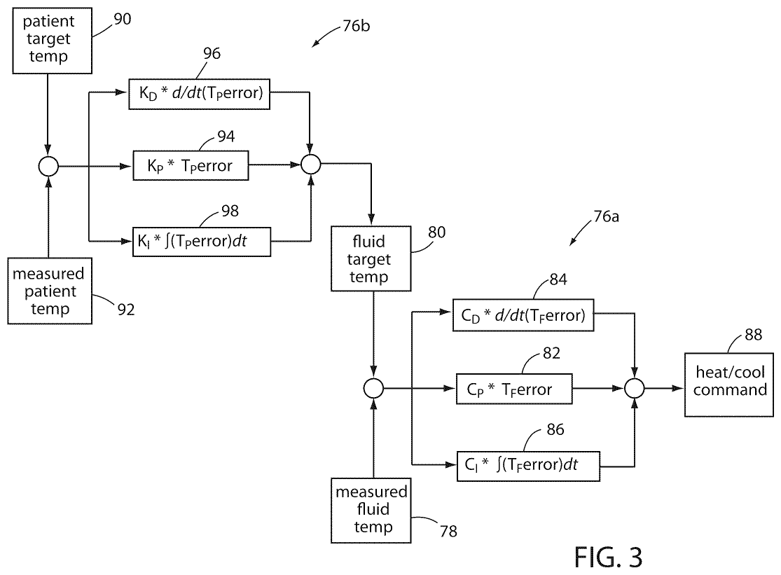

The flow of fluid through bypass line 54 is controllable by way of a bypass valve 58 positioned at the intersection of bypass line 54 and outlet manifold 42 (FIG. 3). When open, bypass valve 58 allows fluid to flow through circulation channel 36 to outlet manifold 42, and from outlet manifold 42 to the connected thermal pads 24. When closed, bypass valve 58 stops fluid from flowing to outlet manifold 42 (and thermal pads 24) and instead diverts the fluid flow along bypass line 54. In some embodiments, bypass valve 58 may be controllable such that selective portions of the fluid are directed to outlet manifold 42 and along bypass line 54. In some embodiments, bypass valve 58 is controlled in any of the manners discussed in commonly assigned U.S. patent application Ser. No. 62/610,319 filed Dec. 26, 2017, by inventors Gregory Taylor et al. and entitled THERMAL SYSTEM WITH OVERSHOOT REDUCTION, the complete disclosure of which is incorporated herein by reference.

The incoming fluid flowing into inlet manifold 52 from inlet ports 50 and/or bypass line 54 travels back toward pump 34 and into an air remover 60. Air remover 60 includes any structure in which the flow of fluid slows down sufficiently to allow air bubbles contained within the circulating fluid to float upwardly and escape to the ambient surroundings. In some embodiments, air remover 60 is constructed in accordance with any of the configurations disclosed in commonly assigned U.S. patent application Ser. No. 15/646,847 filed Jul. 11, 2017, by inventor Gregory S. Taylor and entitled THERMAL CONTROL SYSTEM, the complete disclosure of which is hereby incorporated herein by reference. After passing through air remover 60, the circulating fluid flows past a valve 62 positioned beneath fluid reservoir 32. Fluid reservoir 32 supplies fluid to thermal control unit 22 and circulation channel 36 via valve 62, which may be a conventional check valve, or other type of valve, that automatically opens when reservoir 32 is coupled to thermal control unit 22 and that automatically closes when reservoir 32 is decoupled from thermal control unit 22 (see FIG. 2). After passing by valve 62, the circulating fluid travels to pump 34 and the circuit is repeated.

Controller 48 of thermal control unit 22 is contained within main body 30 of thermal control unit 22 and is in electrical communication with pump 34, heat exchanger 40, outlet temperature sensor 44, bypass valve 58, a patient sensor module 64, and a user interface 66. Controller 48 includes any and all electrical circuitry and components necessary to carry out the functions and algorithms described herein, as would be known to one of ordinary skill in the art. Generally speaking, controller 48 may include one or more microcontrollers, microprocessors, and/or other programmable electronics that are programmed to carry out the functions described herein. It will be understood that controller 48 may also include other electronic components that are programmed to carry out the functions described herein, or that support the microcontrollers, microprocessors, and/or other electronics. The other electronic components include, but are not limited to, one or more field programmable gate arrays, systems on a chip, volatile or nonvolatile memory, discrete circuitry, integrated circuits, application specific integrated circuits (ASICs) and/or other hardware, software, or firmware, as would be known to one of ordinary skill in the art. Such components can be physically configured in any suitable manner, such as by mounting them to one or more circuit boards, or arranging them in other manners, whether combined into a single unit or distributed across multiple units. Such components may be physically distributed in different positions in thermal control unit 22, or they may reside in a common location within thermal control unit 22. When physically distributed, the components may communicate using any suitable serial or parallel communication protocol, such as, but not limited to, CAN, LIN, Firewire, I-squared-C, RS-232, RS-465, universal serial bus (USB), etc.

User interface 66, which may be implemented as a control panel or in other manners, allows a user to operate thermal control unit 22. User interface 66 communicates with controller 48 and includes a display 68 and a plurality of dedicated controls 70. Display 68 may be implemented as a touch screen, or, in some embodiments, as a non-touch-sensitive display. Dedicated controls 70 may be implemented as buttons, switches, dials, or other dedicated structures. In any of the embodiments, one or more of the functions carried out by a dedicated control 70 may be replaced or supplemented with a touch screen control that is activated when touched by a user. Alternatively, in any of the embodiments, one or more of the controls that are carried out via a touch screen can be replaced or supplemented with a dedicated control 70 that carries out the same function when activated by a user. In some embodiments, user interface 66 and display 68 are adapted to carry out any of the functions disclosed in commonly assigned U.S. patent application Ser. No. 62/610,362 filed Dec. 26, 2017, by inventor Gregory S. Taylor and entitled THERMAL SYSTEM WITH GRAPHICAL USER INTERFACE, the complete disclosure of which is incorporated herein by reference.

Through either dedicated controls 70 and/or a touch screen display (e.g. display 68), user interface 66 enables a user to turn thermal control unit 22 on and off, select a mode of operation, select a target temperature for the fluid delivered to thermal pads 24, select a patient target temperature, and control other aspects of thermal control unit 22. In some embodiments, user interface 66 may include a pause/event control, a medication control, and/or an automatic temperature adjustment control that operate in accordance with the pause event control 66b, medication control 66c, and automatic temperature adjustment control 66d disclosed in commonly assigned U.S. patent application Ser. No. 62/577,772 filed on Oct. 27, 2017, by inventors Gregory Taylor et al. and entitled THERMAL SYSTEM WITH MEDICATION INTERACTION, the complete disclosure of which is incorporated herein by reference. Such controls may be activated as touch screen controls or dedicated controls 70.

Patient sensor module 64 (FIG. 2) includes a core patient temperature probe port 72 and a plurality of auxiliary sensor ports 74. Core patient temperature probe port 72 is adapted to couple to a patient temperature probe 73 that is adapted to sense the core temperature of the patient at the location of the sensor. Patient temperature probe 73 is placed at a location on or in the patient's body that provides a measurement of the patient's core temperature, such as, but not limited to, in the patient's esophagus and/or rectum. In one embodiment, the patient temperature probe 73 is a conventional Y.S.I. 400 probe marketed by YSI Incorporated of Yellow Springs, Ohio, or another type of probe that is YSI 400 compliant. In other embodiments, probe 73 may be a different type of core temperature probe. Regardless of the specific type of patient temperature sensor used in thermal control system 20, patient temperature probe 73 is connected to patient sensor module 64 and sensor module 64 forwards the patient core temperature readings to controller 48.

Auxiliary sensor ports 74 are configured to couple to auxiliary links 77 that relay data from one or more auxiliary patient sensors 75 to controller 48 so that controller 48 may utilize the data when controlling the heating and cooling of the fluid delivered to thermal pads 24. Links 77 may be wired or wireless. In the embodiment shown in FIG. 2, patient sensor module 64 includes three auxiliary sensor ports 74 for receiving data from up to three different sensors 75. It will be understood that this number may vary. It will also be understood that the type of data collected by the auxiliary sensors 75 may vary. Indeed, in some embodiments, one or more auxiliary sensors 75 collect data of a first type while one or more other auxiliary sensors 75 collect data of a second type.

In the embodiment shown in FIG. 2, patient sensor module 64 is coupled to a first auxiliary sensor 75a integrated into first thermal pad 24a, a second auxiliary sensor 75b integrated into second thermal pad 24b, and a third auxiliary sensor 75c integrated into third thermal pad 24c. It will be understood that this is merely one arrangement from a virtually unlimited number of arrangements of auxiliary sensors 75. For example, it will be understood that, in some embodiments, none of auxiliary sensors 75 are integrated into any of thermal pads 24a-c, while in other embodiments, one or more of auxiliary sensors 75 are integrated therein. Still further, although FIG. 2 illustrates one sensor 75 integrated into each thermal pad 24, this too can be changed. For example, in some embodiments, multiple auxiliary sensors 75 are integrated into a single thermal pad 24.

Patient auxiliary sensors 75a-c are adapted to detect one or more of the following characteristics of patient 26: a temperature of the patient at an intermediate depth (e.g. subcutaneous) between the patient's core and the patient's skin; a morphological measurement of the patient, such as, but not limited to, a measurement of the patient's thickness and/or a parameter associated with a patient's thickness, such as a Body Mass Index (BMI) or category of BMI's into which the patient falls; a perfusion level of the patient's blood; a surface measurement of the patient's temperature (e.g. a skin temperature measurement); a measurement of the patient's metabolic rate (e.g. a measurement of the patient's end-tidal carbon dioxide (ETCO₂) levels; and/or a bio-impedance measurement of one or more body regions of patient 26.

As was noted previously, patient auxiliary sensors 75a-c may be combined in different manners in thermal control system 20 and may also be integrated into thermal pads 24 and/or used as stand-alone sensors external to thermal pads 24 in different manners from what is shown in FIG. 2. Thus, for example, in some embodiments, a single torso thermal pad 24a may include multiple bio-impedance sensors 75 along with an intermediate temperature sensor 75. Such an embodiment may also include one or more stand-alone auxiliary sensors 75 that are positioned against the palms of the patient and/or the bottom of the patient's feet to measure the patient's temperature (surface and/or intermediate temperatures) at those locations. Such an embodiment may also utilize leg thermal pads 24b and 24c that include no sensors, fewer auxiliary sensors 75 than the torso thermal pad 24a, and/or the same auxiliary sensors 75 as thermal pad 24a. Numerous other examples and configurations are possible.

Controller 48 is adapted to control the thermal therapy applied to the patient in multiple different modes. User interface 66 allows a user to select from these different modes. Although other modes may be implemented, controller 48 is adapted to carry out at least a manual mode and an automatic mode, both of which may be used for cooling and heating the patient. In the manual mode, a user selects a target temperature for the fluid that circulates within thermal control unit 22 and that is delivered to thermal pads 24. Thermal control unit 22 then automatically makes adjustments to heat exchanger 40 in order to ensure that the temperature of the fluid exiting supply hoses 28a is at the user-selected temperature.

In the automatic mode, the user selects a target patient temperature using user interface 66, rather than a target fluid temperature. After selecting the target patient temperature, controller 48 makes automatic adjustments to the temperature of the fluid in order to bring the patient's temperature to the desired patient target temperature. In this mode, the temperature of the circulating fluid may vary as necessary in order to bring about the target patient temperature. In order to carry out the automatic mode, thermal control unit 22 utilizes patient sensor module 64 and, at a minimum, patient temperature readings from patient core temperature probe 73. As will be discussed in greater detail below, controller 48 is adapted in some embodiments to use one or more additional readings from patient auxiliary sensors 75.

FIG. 3 illustrates a pair of feedback loops 76a and 76b that are used in at least one embodiment of thermal control unit 22. Feedback loop 76a is used by controller 48 when thermal control unit 22 is operating in the manual mode and feedback loops 76a and 76b are both used by controller 48 when thermal control unit 22 is operating in the automatic mode. Feedback loop 76a uses a measured fluid temperature 78 and a fluid target temperature 80 as inputs. Measured fluid temperature 78 comes from outlet temperature sensor 44. Fluid target temperature 80, when thermal control unit 22 is operating in the manual mode, comes from a user inputting a desired fluid temperature using user interface 66. When thermal control unit 22 is operating in the automatic mode, fluid target temperature 80 comes from the output of control loop 76b, as discussed more below.

Control loop 76a determines the difference between the fluid target temperature 80 and the measured fluid temperature 78 (Trerror) and uses the resulting error value as an input into a conventional Proportional, Integral, Derivative (PID) control loop. That is, controller 48 multiplies the fluid temperature error by a proportional constant (CP) at step 82, determines the derivative of the fluid temperature error over time and multiplies it by a constant (CD) at step 84, and determines the integral of the fluid temperature error over time and multiplies it by a constant (CI) at step 86. The results of steps 82, 84, and 86 are summed together and converted to a heating/cooling command at step 88. The heating/cooling command is fed to heat exchanger 40 and tells heat exchanger 40 whether to heat and/or cool the circulating fluid and how much heating/cooling power to use.

Control loop 76b which, as noted, is used during the automatic mode, determines the difference between a patient target temperature 90 and a measured patient temperature 92. Patient target temperature 90 is input by a user of thermal control unit 22 using controls 70 and/or display 68 of user interface 66. Measured patient temperature 92 comes from a patient temperature probe 73 coupled to port 72 (FIG. 2). Controller 48 determines the difference between the patient target temperature 90 and the measured patient temperature 92 (T_{P}error) and uses the resulting patient temperature error value as an input into a conventional PID control loop (FIG. 3). As part of the PID loop, controller 48 multiples the patient temperature error by a proportional constant (K_{P}) at step 94, multiplies a derivative of the patient temperature error over time by a derivative constant (K_{D}) at step 96, and multiplies an integral of the patient temperature error over time by an integral constant (K_{I}) at step 98. The results of steps 94, 96, and 98 are summed together and converted to a target fluid temperature value 80. The target fluid temperature value 80 is then fed to control loop 76a, which uses it to compute a fluid temperature error, as discussed above.

It will be understood by those skilled in the art that although FIG. 3 illustrates two PID control loops 76a and 76b, other types of control loops may be used. For example, loops 76a and/or 76b can be replaced by one or more PI loops, PD loops, and/or other types of control equations. Controller 48 implements loops 76a and/or 76b multiple times a second in at least one embodiment, although it will be understood that this rate may be varied widely. In some embodiments, the coefficients used with the control loops may be varied by controller 48 depending upon the patient's temperature reaction to the thermal therapy, among other factors. One example of such dynamic coefficients is disclosed in commonly assigned U.S. patent application Ser. No. 62/577,772 filed on Oct. 27, 2017, by inventors Gregory Taylor et al. and entitled THERMAL SYSTEM WITH MEDICATION INTERACTION, the complete disclosure of which is incorporated herein by reference. Still other modifications of control loops 76a and/or 76b may be made, including the use of feedforward control by controller 48, as will be discussed below in more detail.

After controller 48 has output a heat/cool command at step 88 to heat exchanger 40, controller 48 takes another patient temperature reading 92 and/or another fluid temperature reading 78 and re-performs loops 76a and/or 76b. The specific loop(s) used, as noted previously, depends upon whether thermal control unit 22 is operating in the manual mode or automatic mode.

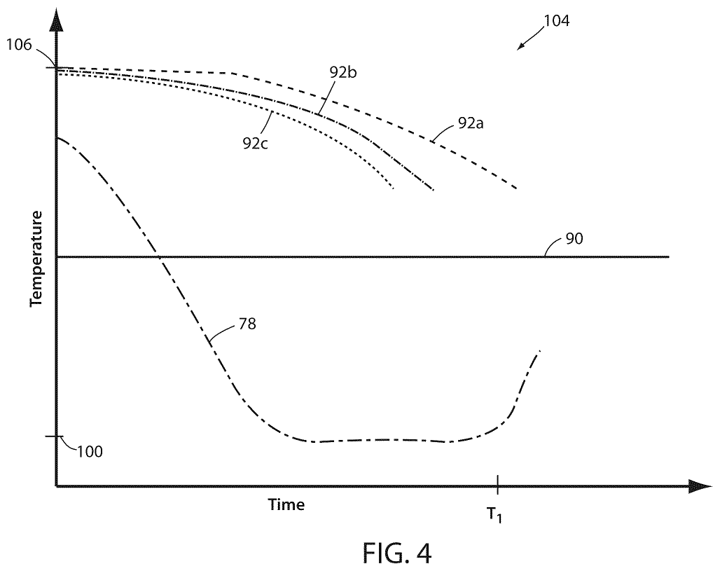

It will also be understood by those skilled in the art that the output of the control loop 76a may be limited such that the temperature of the fluid delivered to thermal pads 24 by thermal control unit 22 never strays outside of a predefined maximum and a predefined minimum. The predefined minimum temperature, an example of which is shown in FIG. 4 and assigned the reference number 100, is a temperature below which controller 48 does not lower the temperature of the circulating fluid. Minimum temperature 100 is designed as a safety temperature and may vary. In some embodiments, it may be set to about four degrees Celsius, although other temperatures may be selected. The predefined maximum temperature (not shown) is a temperature above which controller 48 does not heat the circulating fluid. The predetermined maximum temperature is also implemented as a safety measure and may be set to about forty degrees Celsius, although other values may be selected.

In at least one embodiment of thermal control unit 22, controller 48 is configured to utilize information from one or more patient auxiliary sensors 75 that provide information about the patient's morphology, as well as core patient temperature readings from temperature probe 73. Depending upon the type and/or number of auxiliary sensors 75 used in a particular embodiment, controller 48 uses the information from the auxiliary sensors 75 in different manners. In at least one embodiment of thermal control system 20, thermal control unit 22 is coupled to one or more bio-impedance sensors 75 that are adapted to take bio-impedance readings from the patient. The bio-impedance readings measure the electrical impedance of one or more portions of the patient's body. The bio-impedance readings are used to estimate a size or thickness of the patient. The size or thickness may be measured and/or quantified in a number of different manners, such as, but not limited to, the following: a Body Mass Index (BMI) of the patient; a Total Body Water (TBW) estimate of the patient; a fat-free body mass estimate of the patient; and/or a body fat estimate of the patient.

When sensor 75 is implemented to sense bio-electric impedance, it may be implemented using conventional bioelectrical impedance analysis, and may use any known techniques for analyzing a patient's body composition, including, but not limited to, transmitting electrical signals of multiple frequencies through the patient's body. In some embodiments, the bio-impedance sensor uses a pair of electrodes positioned at different areas of the patient's body, such as, but not limited to, a first electrode positioned adjacent a patient's wrist and a second electrode positioned adjacent the patient's contralateral ankle. In other embodiments, the electrodes are positioned to focus more on the body composition of the patient's torso and/or legs, rather than the rest of the patient's body.

As noted, in some embodiments, controller 48 uses the bio-impedance reading(s) from sensor 75 to make an estimate of the patient's BMI. In some of such embodiments, controller 48 estimates which category of BMI readings that patient falls into, rather than an individual BMI value. For example, in one embodiment, controller 48 is configured to estimate whether the patient falls into three categories of BMI readings: patients having BMIs less than 25, patients with BMI values of 25 to 35, and patients with BMI values above 35. In other embodiments, different ranges of BMI readings may be used, and fewer or greater numbers of these ranges may also or alternatively be used. In such embodiments, auxiliary sensors 75 do not need to be able to accurately correlate their readings to a specific BMI value, but instead only correlate their readings to a plurality of BMI ranges.

Controller 48 uses the estimated BMI range of the patient when controlling heat exchanger 40 and carrying out thermal treatment of the patient. In at least one embodiment, controller 48 uses a first set of coefficients (C_{P}, C_{D}, C_{I}, K_{P}, KD, and K_{I}) for control loops 76a and/or 76b when applying thermal therapy to a patient whose BMI falls within a first range of BMI values; a second set of coefficients for control loops 76a and/or 76b when applying thermal therapy to a patient whose BMI falls within a second range of BMI values; a third set of coefficients for control loops 76a and/or 76b when applying thermal therapy to a patient whose BMI falls within a third range of BMI values; and so on. The multiple sets of coefficients may include six coefficients that are different from all six of the coefficients in the other sets, or it may include less than six coefficients that are different from those of another one of the sets of coefficients. Further, as noted, in some embodiments controller 48 implements either or both of loops 76a and 76b using less than three coefficients each (e.g. a PI control loop, a PD control loop, etc.), and in such embodiments, the differing coefficients in each set will be less than six.

The different coefficients used by controller 48 for controlling heat exchanger 40 are chosen such that they better match the type of patient being thermally treated. That is, patients with large BMI's tend to take longer to have their core temperature adjusted and react more slowly to changes in the temperature of the fluid circulating through thermal pads 24, as compared to patients with lower BMIs. Accordingly, controller 48 uses coefficients that better match the type of patient undergoing thermal treatment by thermal control system 20.

In some embodiments, adjustments to other control aspects are made by controller 48 in response to different patient BMI ranges. Such adjustments are made in addition to using different coefficients or are made as an alternative to using different coefficients, depending upon the particular embodiment. Such adjustments include, but are not limited to, using different limits of integration in either or both of the integral terms (C_{I} and K_{I}); using different limits for any one or more of the error terms (T_{P}error, T_{P}error); executing either or both of loops 76a, 76b at different rates; utilizing feedforward controls aspects in addition to feedback information to control heat exchanger 40; and using a different algorithm to convert the sum from steps 82, 84, and 86 to a heating/cooling command at step 88.

In still other embodiments, the particular BMI range in which a particular patient falls causes controller 48 to utilize, not just one set of coefficients, but a group of coefficients sets with control loops 76a and/or 76b. A first set of coefficients from the group is used during initial treatment of the patient and one or more of the other sets from the group are used at later stages of the thermal treatment (e.g. when the patient's measured temperature 92 nears, or reaches, the patient target temperature 90). For each defined BMI range, controller 48 selects a group of coefficient sets. Controller 48 therefore correlates not just a single set to each range of BMI readings, but multiple sets. In some embodiments, the only change controller 48 makes in response to the BMI range in which a particular patient falls is the selection of a particular group of coefficients. In other embodiments, controller 48 makes other changes to its control of heat exchanger 40 in response to differing patient BMI ranges. Such other changes may include any of those mentioned above (e.g. changing limits of integration, error values, loop frequencies, command algorithms, and/or adding feedforward control).

As an alternative to using bio-impedance readings to estimate a BMI range of a patient, thermal control system 20 may be modified to include alternative or additional auxiliary sensors 75 that estimate a patient's thickness and/or BMI range using different technologies. One such alternative sensor 75 is a near infrared sensor that emits near infrared waves into a portion of the patient's body and detects scattering levels of the near infrared waves. The infrared waves are tuned to a frequency (or frequencies) that are absorbed by fat in the patient's body. By detecting different levels of infrared scatter (corresponding to different amounts of fat absorbance), the sensor is able to determine fat levels within patients' bodies. Sensor 75 forwards the detected fat levels to controller 48, which then utilizes them in a similar manner to how controller 48 utilizes the thickness and/or BMI readings, as discussed above. That is, controller 48 alters its control of heat exchanger 40 based upon how much fat is detected by the infrared sensor 75. The alterations include any of the alternations discussed above with respect to different thickness and/or BMI ranges (e.g. switching coefficients, integration limits, etc.).

When thermal control system 20 includes a near infrared sensor 75 adapted to detect fat levels within a patient's tissue, the infrared sensor 75 can be constructed generally in the same manner as the sensor assembly 12 disclosed in commonly assigned U.S. patent application Ser. No. 14/708,383 filed May 11, 2015, by inventors Marko Kostic et al. and entitled TISSUE MONITORING APPARATUS AND SYSTEM, the complete disclosure of which is incorporated herein by reference. Sensor assembly 12 may be adjusted from the embodiments disclosed in that application by selecting a frequency (or set of frequencies) of infrared light that, instead of primarily detecting different chromophore concentrations, are more suitable for detecting different fat concentrations (or substances that are proxies for varying fat concentrations, such as, but not limited to, water concentrations).

In addition to the bio-impedance and near infrared sensors 75 mentioned above, thermal control system 20 can be utilized with other types of auxiliary sensors 75 that measure information about a patient's morphology, such as, ultrasonic sensors, perfusion sensors, and others. The morphological information that is measured includes, but is not limited to, the patient's BMI range, fat content, water content, abdominal thickness (or other thickness), or another parameter indicative of the amount and/or type of tissue between the patient's exterior and core. Such measurements provide controller 48 with data indicative of how resistive the patient's tissue will be to thermal transfer between the patient's core and the patient's exterior. This allows controller 48 to control the temperature of the fluid delivered to thermal pad 24 more effectively so as to bring the patient to the desired target temperature in less time and/or with less overshoot. In other words, this data provides controller 48 with information regarding how difficult or how easy it will be for the heat or cold applied to the patient's exterior via thermal pads 24 to penetrate to the patient's core. By knowing this information, controller 48 is able to adjust its control of heat exchanger 40 to accommodate the patient's morphology.

Accordingly, people skilled in the art will recognize that controller 48 may use many other measurements beside BMI or BMI ranges for determining a patient's likely resistance to thermal treatment. BMI is conventionally determined by measurements of a patient's weight and height, and provides a generalized measure of a patient's thickness or thinness. The measurement, however, is only a general measure and tends to assign taller people a higher BMI than shorter people having the same thickness. Further, BMI is an overall measurement for the patient's entire body, which may or may not necessarily correspond to the thickness of the patient's body at the location(s) where thermal pads 24 are applied. Controller 48 is therefore configured to work with readings from sensor(s) 75 that are not necessarily BMI readings, or that don't necessarily correlate precisely to BMI readings (although controller 48 can, of course, also use BMI readings). Instead, controller 48 is configured to work with any data regarding the patient's thickness and/or fat content in those area(s) where a thermal pad 24 is applied to the patient. As a result, controller 48 may use bio-impedance readings from a bio-impedance sensor 75 and/or readings from an infrared sensor 75 without performing any additional computation that seeks to convert those readings to a BMI level, or a category of BMI levels. Such readings provide controller 48 with sufficient information to adjust the control of heat exchanger 40 without requiring any such additional computation.

It will be understood that, although controller 48 does not need to calculate an actual BMI value, or determine a BMI category to which the patient belongs, it can still be configured to do so. Indeed, in at least one embodiment, controller 48 calculates the patient's BMI based upon actual weight and height readings of the patient. In such embodiments, the patient's height and weight may be input by a caregiver using user interface 66, or it may be communicated to controller 48 via a wireless or wired interface built into thermal control unit 22. The interface communicates with one or more devices adapted to measure the patient's height and/or weight, or one or more devices having such data stored therein (e.g. an Electronic Medical Record and/or a headwall unit having patient data stored therein, such as disclosed in commonly assigned U.S. patent application Ser. No. 62/600,000 filed Dec. 18, 2017, by inventors Alexander Bodurka and entitled SMART HOSPITAL HEADWALL SYSTEM, the complete disclosure of which is incorporated herein by reference). In some embodiments, thermal control unit 22 includes an interface adapted to communicate (wired or wirelessly) with a patient support apparatus 102 (FIG. 1) having a scale built therein. Patient support apparatus 102 is used to support the patient while he or she is undergoing thermal treatment and measures the patient's weight while the patient is supported thereon. When calculating an actual BMI value for a patient, controller 48 may thereafter carry out thermal treatment of the patient without utilizing any auxiliary sensors 75. In other embodiments, controller 48 may use the BMI value, patient core temperature readings, and outputs from auxiliary sensors 75 to control heat exchanger 40.

In some embodiments, where sensors 75 adapted to measure patient variables indicative of the patient's morphology, the sensors 75 are included within torso thermal pad 24a and positioned along the sides of torso pad 24a so that the sensors 75 detect morphological values based on readings from the side(s) of the patient's chest and/or sides of the patient's abdomen. In other embodiments, sensors 75 may be integrated into pad 24a at different locations. In some embodiments, one or more sensors 75 are included that are separate from pads 24 so that the sensor(s) 75 can be applied at a specific location on the patient's body independent of the thermal pad's conformance to that particular patient's body. In this manner, the sensor(s) 75 can be more consistently applied at a common location for all patient's undergoing thermal treatment with thermal control system 20.

The manner in which controller 48 uses sensor(s) 75 may be better understood with respect to FIG. 4. FIG. 4 shows a temperature graph 104 illustrating how patients having different morphologies-such as different BMI levels and/or thicknesses-generally respond differently to thermal treatment. As shown therein, graph 104 includes a set of fluid temperature readings 78 indicating the temperature of fluid supplied to thermal pads 24, and first, second, and third sets of patient temperature readings 92a, 92b, and 92c, respectively. Graph 104 further includes a target patient temperature 90 and a minimum fluid temperature 100 for the fluid supplied to thermal pads 24.

First set of patient temperature readings 92a corresponds to a patient having a relatively thick morphology and third set of patient temperature readings 92c corresponds to a patient having a relatively thick morphology. Second set of patient temperature readings 92b corresponds to a patient having a morphology between those of readings 92a and 92c. As can be seen in graph 104, when all other factors are held equal, the thicker the patient, the longer it takes for the patient's temperature to approach the target temperature 90. This is because the greater amount of patient tissue creates more thermal insulation, and that thermal insulation resists the transfer of heat to/from the patient's exterior to the patient's core. Thermal pads 24a-c therefore take longer to affect the patient's core temperature. As a result, when all other factors are equal, a patient having a greater degree of thickness will have a greater lag between the time a hot or cold temperature is applied to the patient via thermal pads 24 and the time the hot or cold temperature changes the patient's core temperature.

Controller 48, in some embodiments, uses the variations in this lag time when deciding when to switch from heating the fluid to cooling the fluid, or vice versa. In the example shown in FIG. 4, controller 48 is cooling a patient from an initial temperature 106 to target temperature 90 and cools the fluid circulating through thermal control unit 22 and pads 24 to minimum temperature 100. Controller uses the lag time to decide when to start warming this circulating fluid. This moment is identified in FIG. 4 as T₁. In order to reduce overshoot, controller 48 is configured to choose moment T₁ sooner for thicker patients, and to choose moment T₁ later for thinner patients. This is because the heat that is going to be applied through thermal pads 24 will take longer to affect the patient's core for the thicker patients than for the thinner patients. Consequently, in order to stop the patient's temperature at target temperature 90 and prevent it from continuing past temperature 90, controller 48 must respond sooner with heat when treating thicker patients because the heat will take longer to penetrate to the patient's core. Controller 48 is therefore configured to use measurements of the patient's thickness from one or more auxiliary sensors 75 when determining when to choose moment T₁. Similarly, controller 48 may also be configured to use measurements of the patient's thickness from one or more auxiliary sensors 75 when determining future transitions from heating to cooling, and vice versa, that occur after T₁.

In some embodiments, controller 48 is configured to use the patient thickness readings to control heat exchanger 40 in a feedforward method. The feedforward method may be used alone or in combination with feedback control (e.g. feedback loops 76a and 76b). When using feedforward control, controller 48 is programmed with a mathematical model of how variations in patient thicknesses affect the patient's response to thermal therapy. The mathematical model may be built upon empirical data gathered from multiple thermal therapy sessions applied to patients of different morphologies. Other constructions are possible as well. Controller 48 uses the model and measurements from the thickness sensor(s) 75 to adjust its commands to heat exchanger 40 so that thermal control unit 22 accounts for these differences in a manner that allows thermal control unit 22 to expeditiously bring the patient to target temperature 90 while avoiding undue overshoot.

In addition to, or in lieu of, auxiliary sensors 75 that measure parameters indicative of the patient's thickness, thermal control system 20 is also equipped in some embodiments with one or more auxiliary sensors 75 that measure the patient's temperature at an intermediate location. The term “intermediate location” refers to a portion of the patient's body that is deeper than the patient's surface temperature, but is not deep enough to reach the patient's core. As will be discussed in more detail below, controller 48 uses this intermediate temperature measurement to better assess how quickly the thermal treatment being applied to the patient's surface via thermal pads 24 is penetrating into the interior of the patient's body. This speed information is then used when controlling heat exchanger 40.

In at least one embodiment, thermal control system 20 includes one or more intermediate temperature sensors that are implemented as perfusion sensors. The perfusion sensors monitor the amount of blood in the patient's tissue, which generally changes in response to the patient's temperature. Such changes include lower perfusion levels when the patient is cold and higher perfusion levels when the patient is warm. In some embodiments, the perfusion sensor is positioned to measure perfusion levels in the patient's palms and/or the sole(s) of the patient's foot/feet. Such locations may be chosen because they are high thermal exchange areas of a patient's body. It will be understood, however, that the perfusion sensor(s) 75 may be positioned at other locations, including, but not limited to, any suitable location on the patient's torso. In some embodiments, one or more perfusion sensors are built into one or more of the thermal pads 24a-c.

Although any conventional perfusion sensor may be used with thermal control unit 22, in some embodiments, one or more perfusion sensors of the type disclosed in commonly assigned U.S. patent application Ser. No. 14/708,383 filed May 11, 2015, by inventors Marko Kostic et al. and entitled TISSUE MONITORING APPARATUS AND SYSTEM, the complete disclosure of which is incorporated herein by reference. As was noted previously, the sensors disclosed in that patent application may be tuned to measure fat levels in a patient's tissue instead of perfusion levels. It will be understood, however, that in some embodiments of thermal control system 20, a perfusion sensor of the type disclosed in the '383 application may be tuned to detect both perfusion levels and fat levels, or it may be configured to switch back and forth between measuring fat and perfusion levels so that controller 48 is apprised of both values from a single sensor. Still further, perfusion sensors of the type disclosed in the '383 application may utilize a plurality of detectors positioned at different locations with respect to an emitter in order to detect perfusion, fat, and/or other characteristics at specific depths and/or ranges of depths. Still other variations are possible.

In other embodiments, one or more intermediate temperature readings may be supplied by an ultrasonic sensor 75 adapted to perform acoustic thermometry. Although acoustic thermometry tends to detect a temperature reading over the entire area in which the detected sound wave travels, such readings are still useful to controller 48, particularly when they are combined with a temperature reading from one or more surface temperature readings that measure the patient's temperature at his or her surface (e.g. skin). By knowing the surface temperature, the depth of penetration of the acoustic waves, and the temperature readings generated from the acoustic waves, controller 48 is able to determine the extent to which the surface temperature extends into intermediate regions of the patient's body. Controller 48 is therefore configured in some embodiments to use such surface temperature readings in combination with acoustic thermometer readings to determine the degree of penetration of the skin temperature to intermediate locations within the patient's body.

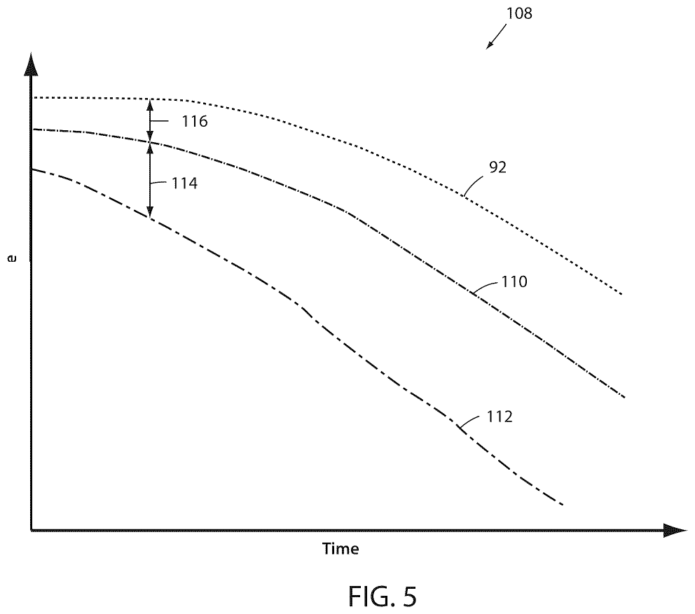

FIG. 5 shows a graph 108 illustrating how surface, intermediate, and core patient temperature readings may vary during the course of thermal treatment, and how this variation is used by at least one embodiment of thermal control unit 22 when carrying out thermal treatment of a patient. As shown therein, graph 108 includes a set of core patient temperature readings 92, a set of intermediate patient temperature readings 110, and a set of surface patient temperature readings 112. Although the example shown in FIG. 5 shows readings taken over a period of time when a patient is being cooled by thermal control unit 22, it will be understood that such readings are also taken and monitored by controller 48 during times when the patient is being warmed.

Core temperature readings 92 come from patient temperature probe 73. Intermediate temperature readings 110 come from one or more intermediate temperature sensors 75, such as, but not limited to, the acoustic thermometer sensors and/or perfusion sensors mentioned above. Surface temperature readings 112 come from one or more conventional surface temperature sensors 75 that are positioned in contact with the patient's skin and that feed their outputs into patient sensor module 64.

Controller 48 is configured in at least one embodiment to monitor a surface-intermediate temperature difference 114 and an intermediate-core temperature difference 116 during the thermal treatment of a patient (FIG. 5). Controller 48 monitors these variables 114 and 116 in order to determine how fast the thermal effects of thermal pads 24 are penetrating the patient's body. This allows controller 48 to determine how responsive the patient is to the thermal treatment and to choose when to transition from cooling to heating (e.g. moment T₁ in FIG. 4), or vice versa, in a manner that helps to reduce temperature overshoot. More particularly, controller 48 monitors variables 114 and 116 during thermal treatment of a patient and uses these variables to predict when the patient's core temperature will reach target temperature 90. Controller 48 may be programmed to make these predictions in various manners.

In some embodiments, controller 48 predicts when a patient's core temperature will reach the target temperature 90 by comparing the value of variable 114 with respect to the rate of change of variable 116, and/or by comparing the rate of change of variable 114 with respect to the rate of change of variable 116. These comparisons provide an indication of how fast the removal of heat (or addition of heat, as the case may be) from the patient's surface via thermal pads 24 is translating into a removal of heat from the patient's core. These comparisons also provide an indication of how long of a lag there is between the surface temperature affecting the core temperature. By repetitively monitoring these variables 114 and 116, either alone or in combination with each other, controller 48 also calculates how long it will likely take for heat applied to the patient's skin to impact the patient's core (and/or for cold applied to the patient's skin to impact the patient's core). Using this calculation, controller 48 is able to determine when to start warming the fluid so that overshoot is reduced or eliminated.

In addition to using variables 114 and 116, controller 48 also uses other factors to determine when a patient will likely reach the target temperature 90 and/or when to transition from heating the circulating fluid to cooling the circulating fluid. These other factors include the slope of the core temperature readings 92; the difference between the core temperature 92 and the target temperature 90; whether medication has been given to the patient; empirical data gathered from previous thermal therapy sessions with patients where surface, intermediate, and core temperature readings were taken; and/or other factors. Indeed, controller 48 is configured in some embodiments to use any of the data and algorithms disclosed in the following commonly assigned U.S. patent applications when determining when a patient's core temperature will reach target temperature 90, and/or when to transition from heating the circulating fluid to cooling the circulating fluid, and vice versa, in order to reduce overshoot: U.S. patent application Ser. No. 62/610,334, filed Dec. 26, 2017, by inventors Gregory Taylor et al. and entitled THERMAL CONTROL SYSTEM; U.S. patent application Ser. No. 62/577,772 filed on Oct. 27, 2017, by inventors Gregory Taylor et al. and entitled THERMAL SYSTEM WITH MEDICATION INTERACTION; and U.S. patent application Ser. No. 62/610,319 filed Dec. 26, 2017, by inventors Gregory Taylor et al. and entitled THERMAL SYSTEM WITH OVERSHOOT REDUCTION, the complete disclosures of all of which are incorporated herein by reference.

In one embodiment of thermal control unit 22, controller 48 is configured to monitor perfusion levels of a patient, such as at the patient's palms or soles of the patient's feet, and look for sudden changes. Such sudden changes are reflective of the patient's body changing its shunting of blood either to or away from these areas, and such changes are typically accompanied by changes in temperature. Controller 48 is configured in at least one embodiment to switch to using a different set of coefficients in loops 76a and/or 76b in response to detecting such a sudden change in perfusion levels in the patient's palms and/or feet.

In still other embodiments, thermal control unit 22 includes one or more thermal cameras as auxiliary sensors 75. Such thermal cameras report temperature readings to controller 48 of the patient's temperature and controller 48 uses the temperature readings when controlling heat exchanger 40. Controller 48 uses the temperature readings from the thermal cameras in any of the manners discussed above. In some embodiments, the thermal cameras are the same as and/or operate in the same manners as disclosed in commonly assigned U.S. Pat. No. 9,814,410 issued Nov. 14, 2017, to inventors Marko N. Kostic et al. and entitled PERSON SUPPORT APPARATUS WITH POSITION MONITORING, the complete disclosure of which is incorporated herein by reference.

In at least one embodiment, at least one of the auxiliary inputs 74 is adapted to receive sensor readings from an end-tidal carbon dioxide (ETCO₂) sensor coupled to the patient undergoing thermal treatment. In this embodiment, the ETCO₂ sensor is incorporated into a mask, or other apparatus, that captures and/or samples the amount of carbon dioxide in the exhaled breath of the patient. The ETCO₂ sensor may utilize one or more infrared sensors to detect the ETCO₂ levels of the patient, or it may use other technologies for measuring the ETCO₂ levels. The auxiliary port 74 that is dedicated to receiving the ETCO₂ level readings forwards the readings to controller 48. Controller 48, in turn, uses the readings to perform one or more of the following actions, depending upon the particular embodiment: (1) determine an indicator of the patient's metabolic activity, such as by determining the volume carbon dioxide exhaled by the patient over a given time period (e.g. per minute); (2) display the ETCO₂ levels (and/or the indicator) on display 68 of user interface 66; (3) adjust the heating/cooling commands sent to heat exchanger 40; (4) adjust a flow rate of the fluid delivered to thermal pads 24; (5) change one or more of the coefficients discussed above in the control loops 76a and/or 76b; and/or (6) adjust a reservoir valve that, as discussed below with respect to commonly assigned U.S. patent application Ser. No. 62/610,319, controls the inclusion and exclusion of reservoir 32 from the circulation channel 36 (e.g. controls when fluid circulating in circulation channel 36 is diverted into reservoir 32, rather than bypassing reservoir 32).

In those embodiments where controller 48 is adapted to adjust the heating and/or cooling commands sent to heat exchanger 40 based on the ETCO₂ readings, controller 48 is programmed to increase the cooling (assuming thermal control unit 22 is being used to cool the patient) in response to an increase in ETCO₂ readings, and to do so earlier than it otherwise would in those embodiments where no ETCO₂ readings are utilized. Such increases provide an early indication that the patient is increasing his or her heat output, and by increasing the cooling in response to such increases, thermal control unit 22 is better able to counteract the increased heating, and thereby better maintain the patient at the desired temperature or more quickly bring the patient to the desired temperature. Alternatively, if the ETCO₂ readings decrease, this provides an indication that the patient's heat output is decreasing, and controller 48 is programmed to decrease the cooling (assuming thermal control unit 22 is being used to cool the patient) in response to such decreases in ETCO₂ readings, and to do so earlier than it otherwise would in those embodiments where no ETCO₂ readings are used. This helps avoid overcooling the patient beyond the patient's target temperature. If thermal control unit 22 is being used to warm a patient, rather than cool the patient, controller 48 may be programmed to take the following actions: decrease the heating in response to an increase in ETCO₂ levels, and increase the heating in response to a decrease in ETCO₂ levels.

It will be understood that thermal control unit 22 can operate in a wide variety of different manners depending upon which specific auxiliary sensors 75 are coupled to patient sensor module 64. In some embodiments, thermal control unit 22 operates only with auxiliary sensors 75 that detect a level of thickness of the patient, such as discussed above with respect to FIG. 4. In other embodiments, thermal control unit 22 operates only with auxiliary sensors 75 that detect one or more intermediate temperatures of the patient and operate in the manners described above with respect to FIG. 5. In still other embodiments, thermal control unit 22 includes at least one sensor 75 that measures the patient's thickness and at least one sensor that measures an intermediate temperature of the patient. In these embodiments, controller 48 uses both the patient's thickness and intermediate temperature readings to determine how quickly to heat or cool the patient, when to transition from heating to cooling, or vice versa, in order to reduce overshoot, and for any of the other purposes discussed above.

It will be understood that thermal control unit 22 can be modified in still other manners from what has been shown and described herein in a variety of other manners. For example, thermal control unit 22 may also be modified to include one or more flow sensors that measure the rate of fluid flow and report this information to controller 48. In such modified embodiments, controller 48 uses the flow rate in determining what heating/cooling commands to send to heat exchanger 40 and/or what flow rate signals to send to pump 34.

The particular order of the components along circulation channel 36 of thermal control unit 22 may also or alternatively be varied from what is shown in FIG. 2. For example, although FIG. 2 depicts pump 34 as being upstream of heat exchanger 40 and air separator 60 as being upstream of pump 34, this order may be changed. Air remover 60, pump 34, heat exchanger 40 and reservoir 32 may be positioned at any suitable location along circulation channel 36. Indeed, in some embodiments, reservoir 32 is moved so as to be in line with and part of circulation channel 36, rather than external to circulation channel 36 as shown in FIG. 2, thereby forcing the circulating fluid to flow through reservoir 32 rather than around reservoir 32.

Further details regarding the construction and operation of embodiments of thermal control unit 22 that are not described herein are found in commonly assigned U.S. patent application Ser. No. 14/282,383 filed May 20, 2014, by inventors Christopher Hopper et al. and entitled THERMAL CONTROL SYSTEM, the complete disclosure of which is incorporated herein by reference.

Thermal control unit 22 may also be modified to include a reservoir valve that is adapted to selectively move fluid reservoir 32 into and out of line with circulation channel 36. The reservoir valve may be positioned in circulation channel 36 between air remover 60 and valve 62. When the reservoir valve is open, fluid from air remover 60 flows along circulation channel 36 to pump 34 without passing through reservoir 32. When the reservoir valve is closed, fluid coming from air remover 60 flows into reservoir 32, and from reservoir 32 the fluid flows back into circulation channel 36 via valve 62. Once back in circulation channel 36, the fluid flows to pump 34 and is pumped to the rest of circulation channel 36 and thermal pads 24 and/or bypass line 54. In some embodiments, the reservoir valve is either fully open or fully closed, while in other embodiments, the reservoir valve may be partially open or partially closed. In either case, the reservoir valve is under the control of controller 48. Further details of such a reservoir valve are disclosed in commonly assigned U.S. patent application Ser. No. 62/610,319 filed Dec. 26, 2017, by inventors Gregory Taylor et al. and entitled THERMAL SYSTEM WITH OVERSHOOT REDUCTION, the complete disclosure of which is incorporated herein by reference.

Thermal control unit 22 may also be modified to include a reservoir temperature sensor that reports its temperature readings to controller 48. Controller 48 uses these temperature readings to decide when to include and exclude reservoir 32 from circulation channel 36 (i.e. when to open and close the reservoir valve discussed above). In some embodiments, controller 48 utilizes a temperature control algorithm to control the reservoir valve using temperature measurements from the reservoir sensor that is the same as the temperature control algorithm 160 disclosed in commonly assigned U.S. patent application Ser. No. 62/577,772 filed on Oct. 27, 2017, by inventors Gregory Taylor et al. and entitled THERMAL SYSTEM WITH MEDICATION INTERACTION, the complete disclosure of which is incorporated herein by reference. In other embodiments, controller 48 utilizes a different control algorithm.

Various additional alterations and changes beyond those already mentioned herein can also be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

What is claimed is:

1. A thermal control unit for controlling a patient's temperature, the thermal control unit comprising:
a fluid outlet adapted to fluidly couple to a fluid supply line of a thermal pad, the thermal pad adapted to be wrapped around a portion of the patient's body;
a fluid inlet adapted to fluidly couple to a fluid return line of the thermal pad;
a circulation channel coupled to the fluid outlet and the fluid inlet;
a pump for circulating fluid through the circulation channel from the fluid inlet to the fluid outlet;
a heat exchanger adapted to add or remove heat from the fluid circulating in the circulation channel;
a fluid temperature sensor adapted to sense a temperature of the fluid;
a patient temperature probe port adapted to receive patient core temperature readings from a patient temperature probe;
a perfusion sensor port adapted to receive sensor readings from a perfusion sensor adapted to detect a patient's blood perfusion levels at at least one of a palm or a foot of the patient; and
a controller in communication with the patient temperature probe port, the pump, the fluid temperature sensor, and the perfusion sensor port, the controller adapted to control the heat exchanger based on both the patient core temperature readings and the patient's blood perfusion levels from at least one of the patient's palm or foot.

2. The thermal control unit of claim 1 wherein the controller is adapted to use a feedback control loop with a first set of coefficients to control the heat exchanger when the sensor readings from the perfusion sensor meet a first criteria and to use the feedback control loop with a second and different set of coefficients to control the heat exchanger when the sensor readings from the perfusion sensor meet a second criteria.

3. The thermal control unit of claim 1 wherein the controller is further adapted to control the heat exchanger such that the patient core temperature readings move toward a target patient temperature.

4. The thermal control unit of claim 3 wherein the controller uses the patient's blood perfusion levels to control the heat exchanger in a feedforward manner.

5. The thermal control unit of claim 1 wherein the perfusion sensor is integrated into the thermal pad.

6. The thermal control unit of claim 1 further including an auxiliary sensor port adapted to receive sensor readings from an auxiliary sensor.

7. The thermal control unit of claim 6 wherein the auxiliary sensor is an intermediate temperature sensor positioned at an exterior location of the patient's body and adapted to measure a temperature of the patient at an intermediate depth between the patient's skin and the patient's core.

8. The thermal control unit of claim 7 wherein the controller is adapted to monitor a lag time between external application of a cold temperature to the patient's skin via the thermal pad and propagation of the cold temperature to the intermediate depth.

9. The thermal control unit of claim 8 wherein the controller is adapted to use the lag time to determine when to transition from cooling the fluid to heating the fluid.

10. The thermal control unit of claim 8 wherein the controller is adapted to use the lag time to predict when the patient core temperature readings will reach a target patient temperature.

11. The thermal control unit of claim 6 wherein the auxiliary sensor is a bio-impedance sensor adapted to detect electrical impedance of the patient.

12. The thermal control unit of claim 6 wherein the auxiliary sensor is an ultrasonic sensor adapted to detect attenuation levels of ultrasonic waves traveling through at least a portion of the patient's body.

13. The thermal control unit of claim 6 wherein the auxiliary sensor is a near infrared sensor adapted to detect attenuation levels of near infrared waves traveling through at least a portion of the patient's body.

14. The thermal control unit of claim 6 wherein the auxiliary sensor port is adapted to receive sensor readings from a plurality of non-temperature sensors, and the controller is further adapted to control the heat exchanger based on the sensor readings from the plurality of non-temperature sensors.

15. The thermal control unit of claim 14 wherein the plurality of non-temperature sensors are integrated into thermal pad.

16. The thermal control unit of claim 6 wherein the auxiliary sensor is an acoustic thermometer.

17. The thermal control unit of claim 6 wherein the auxiliary sensor is adapted to measure a subcutaneous temperature of the patient.
